(19) United States
(12) Patent Application Publication
(10) Pub. No.: US 2015/0121570 A1
(43) Pub. Date: Apr. 30, 2015

(54) GENE IMPLICATED IN DROUGHT STRESS TOLERANCE AND TRANSFORMED PLANTS WITH THE SAME

(71) Applicants: Woo Taek KIM, Gyeonggi-do (KR); Eun Yu KIM, Seoul (KR); Moon Yong RYU, Seoul (KR); Yoon A CHOI, Incheon (KR)

(72) Inventors: Woo Taek KIM, Gyeonggi-do (KR); Eun Yu KIM, Seoul (KR); Moon Yong RYU, Seoul (KR); Yoon A CHOI, Incheon (KR)

(73) Assignee: Industry-Academic Cooperation Foundation, Yonsei University, Seoul (KR)

(21) Appl. No.: 14/258,908

(22) Filed: Apr. 22, 2014

(30) Foreign Application Priority Data
Oct. 24, 2013 (KR) 10-2013-0127074

(51) Int. Cl. C12N 15/82; C12N 5/04

(52) U.S. Cl. CPC C12N 15/8201; C12N 5/04; C12N 15/8273
USPC 800/286; 800/278; 435/419; 800/295; 800/285; 800/290

(57) ABSTRACT

The present invention relates to a composition for improving a tolerance to drought and oxidative stress of a plant, a plant transformed with the composition, a method for preparing a plant transformed with the composition, a method for improving a tolerance to a drought and oxidative stress and a composition and method for a germination rate of a plant. The nucleotide sequences of the present invention are involved in the drought and oxidative stress-tolerance and germination capacity, therefore may be effectively used for cultivating the plants with enhanced tolerance against abiotic stress or novel function of rapid growing or bolting.

### CROSS REFERENCE TO RELATED APPLICATIONS

This application claims priority to Korean Patent Application No. 10-2013-0127074, filed on Oct. 24, 2013 in the Korean Intellectual Property Office, the entire disclosure of which is incorporated herein by reference.

### BACKGROUND

1. Field of the Invention

The present invention relates to a gene implicated in drought stress tolerance and transformed plants with the same.

2. Background of Technique

Abiotic stresses such as drought, salt and cold stresses significantly affect on the growth and productivity of plants. Due to their sessile nature, higher plants are constantly faced with various adverse environmental factors, including drought, high salt, heavy metals, cold, heat shock, and ozone, during their whole life span. These abiotic stresses are a limiting factor for the growth and development of crop plants. Water deficiency causes dramatic reduction of crop production globally, and the decreasing availability of fresh water may pose a future threat to humans and higher plants. Plants have diverse defense strategies to enhance their tolerance to transient and long-term water shortages by triggering signaling network pathways and inducing stress-responsive genes. The cellular and genetic defense mechanisms in response to water stress have been widely documented (Shinozaki and Yamaguchi-Shinozaki, 2007). However, for stress tolerance or sensitivity, our knowledge concerning the biological functions of stress-related genes in higher plants is still rudimentary. Therefore, it is important to study the functions of stress responsive genes to increase the productivity and distribution of crop plants.

Throughout this application, various publications and patents are referred and citations are provided in parentheses. The disclosures of these publications and patents in their entities are hereby incorporated by references into this application in order to fully describe this invention and the state of the art to which this invention pertains.

### SUMMARY

The present inventors have made intensive studies to identify genes for improving a tolerance to a drought stress of a plant. As results, the present inventors have discovered that nucleotide sequences encoding the amino acid sequences of SEQ ID NO:3, concretely nucleotide sequences of SEQ ID NO:1, is related to the tolerance to said stresses and that a plant with enhanced stress tolerance may be obtained by suppressing the expression of said nucleotides. In addition, it is further revealed that inhibition of an expression of said nucleotide sequence improves a germination rate of a plant.

Accordingly, it is an aspect of this invention to provide a composition and a method for improving a tolerance to a drought stress of a plant.

It is another aspect of this invention to provide a plant cell or a plant exhibiting improved tolerance to a drought stress, transformed with the composition according to this invention.

It is still another aspect of this invention to provide a method for preparing transformed plant exhibiting improved drought stress tolerance.

It is further aspect of this invention to provide a composition and a method for improving an oxidative stress of a plant.

It is still further aspect of this invention to provide a composition and a method for promoting a germination rate of a plant.

Other aspects and advantages of the present invention will become apparent from the following detailed description together with the appended claims and drawings.

### BRIEF DESCRIPTION OF THE DRAWINGS

FIGS. 1a to 1d represent the identification and characterization of atamdr T-DNA insertion mutant plants. FIG. 1a shows the result of screening of an ABA-insensitive mutant at the germination stage. Wild-type and knock-out mutant seeds were incubated on MS medium for 7 days with or without ABA. FIG. 1b indicates schematic structure of the atamdr allele with the T-DNA insertion. AtAMDR (Arabidopsis thaliana ABA-Mediated Drought Resistance) gene consisting of one exon and one intron (2208 bp), exists as single copy in Arabidopsis thaliana. The triangle indicates the T-DNA, yellow bar indicate coding regions, red bars indicate the 5′ and 3′ untranslated regions, and the solid black line represents introns. Gene-specific (FW1, FW2, RV1, and RV2) and T-DNA-specific (BP) primers used in genotyping and RT-PCR are indicated with arrows. FIGS. 1c and 1d represent genotyping PCR of the atamdr loss-of-function mutant plant, and RT-PCR analysis of AtAMDR in wild-type and atamdr plants, respectively. In RT-PCR analysis of FIG. 1d, Ubiquitin10 (UBC10) transcript levels were used as loading controls.

FIGS. 2a to 2d represent the expression patterns of AtAMDR in response to various abiotic stress conditions. Total RNA was prepared from light-grown, 7-day-old Arabidopsis seedlings, which had been treated with drought (FIG. 2a), cold (FIG. 2b), high salinity (300 mM NaCl) (FIG. 2c) or 100 μM ABA (FIG. 2d). Induction patterns of AtAMDR were investigated by RT-PCR. RD29A was used as representative control gene for drought, cold and salt stress, and Rab18 was used as control gene for ABA.

Total RNA was prepared from 7-day-old Arabidopsis seedlings, which had been treated with drought (1-2 h), cold (4° C. for 6-12 h), high salinity (300 mM NaCl for 1-3 h) or 100 μM ABA (1-3 h). Induction patterns of AtAMDR were investigated by RT-PCR.

FIG. 3a represents the phenotypes of wild-type, atamdr plants in response to ABA during seed germination. Surface-sterilized seeds were imbibed in water for 2 days at 4° C., sown on MS medium supplied with ABA (0, 0.25, 0.5 and 0.75 μM), and incubated for 7 days at 22° C. under the continuous light. Wild-type plant was more sensitive to ABA than AtAMDR knock-out mutants showed reduced sensitivity to ABA.

FIG. 3b represents the quantification of cotyledon greening in response to ABA. Average and SD values were determined from four biological replicates (n=25).

FIGS. 4a to 4c represent stomatal aperture and H₂O₂ production of wild-type, atamdr plants in response to different concentrations of ABA. FIG. 4a shows ABA-induced stomata closure. Light-grown, 4-week-old mature leaves of wild-type and atamdr plants were incubated in stomatal opening solution for 2 h and transferred to solutions containing the 10 μM ABA for 2 h. Stomata on the abaxial surface were observed by light microscopy. FIG. 4b shows stomatal aperture (the ratio of width to length) after 0 and 10 μM ABA treatments. FIG. 4c shows ABA-induced H₂O₂ production analyzed by DAB staining. Leaves from wild-type, atamdr plants were treated with 0 or 100 μM ABA for 2 h and transferred to 100 μg/mL DAB (3,3′-diaminobenzidine-4HCl) solution overnight. The presence and level of H₂O₂ in the leaves were visualized as a dark brown color.

FIG. 5 represents the comparison of drought stress tolerance of atamdr mutant and wild-type. Light-grown, 2.5-week-old wild-type and atamdr mutant plants were further grown for 13 d without watering. Plants were rewatered, and surviving plants were counted 3 d after rewatering.

### DETAILED DESCRIPTION

In one aspect of this invention, there is provided a composition for improving a tolerance to a drought stress of a plant, comprising a nucleotide sequence encoding the amino acid sequence of SEQ ID NO:2.

The present inventors have made intensive studies to identify genes for improving a tolerance to a drought stress of a plant. As results, the present inventors have discovered that nucleotide sequences encoding the amino acid sequences of SEQ ID NO:3, concretely nucleotide sequences of SEQ ID NO:1, is related to the tolerance to said stresses and that a plant with enhanced stress tolerance may be obtained by suppressing the expression of said nucleotides. In addition, it is further revealed that inhibition of an expression of said nucleotide sequence improves a germination rate of a plant.

In previous study, the present inventors obtained 100 different Arabidopsis thaliana T-DNA insertion mutant plants and investigated their phenotypes in the presence of exogenous abscisic acid (ABA), a plant stress hormone. One of these loss-of-function mutants displayed ABA-insensitive phenotypes at the germination stage and was named atamdr (for Arabidopsis thaliana ABA-mediated drought resistance). The present inventors confirmed gene and transcript level of atamdr by genotyping PCR and RT-PCR. Since the atamdr mutant plant was selected based on the ABA insensitive phenotype, the present inventors compared seed germination rates of wild-type, atamdr mutant plants in the different concentrations of ABA. Also, to confirm ABA insensitive phenotype, the present inventors observed stomatas of loss-of-function mutant plant. The stomata of atamdr mutant still remained open in the presence of ABA. The atamdr mutant accumulated lower amounts of hydrogen peroxide in response to exogenous ABA than did wild-type plant, and showed more sensitive phenotype to drought stress than wild-type Arabidopsis. Thus, the present inventors have concluded that AtAMDR acts as positive regulator in response to ABA-mediated drought stress.

According to a concrete embodiment, the nucleotide sequence of this invention comprises the nucleotide sequence of SEQ ID NO:1.

For nucleotides, the variations may be purely genetic, i.e., ones that do not result in changes in the protein product. This includes nucleic acids that contain functionally equivalent codons, or codons that encode the same amino acid, such as six codons for arginine or serine, or codons that encode biologically equivalent amino acids.

Considering biologically equivalent variations described hereinabove, the nucleic acid molecule of this invention may encompass sequences having substantial identity to them. Sequences having the substantial identity show at least 60%, more preferably at least 80%, most preferably at least 90% similarity to the nucleic acid molecule of this invention, as measured using one of the sequence comparison algorithms. Methods of alignment of sequences for comparison are well-known in the art. Various programs and alignment algorithms are described in: Smith and Waterman, Adv. Appl. Math. 2:482(1981); Needleman and Wunsch, J. Mol. Bio. 48:443(1970); Pearson and Lipman, Methods in Mol. Biol. 24: 307-31(1988); Higgins and Sharp, Gene 73:237-44(1988); Higgins and Sharp, CABIOS 5:151-3(1989) Corpet et al., Nuc. Acids Res. 16:10881-90(1988) Huang et al., Comp. Appl. Bio Sci. 8:155-65(1992) and Pearson et al., Meth. Mol. Biol. 24:307-31(1994). The NCBI Basic Local Alignment Search Tool (BLAST) [Altschul et al., J. Mol. Biol. 215:403-10(1990)] is available from several sources, including the National Center for Biological Information (NBCI, Bethesda, Md.) and on the Internet, for use in connection with the sequence analysis programs blastp, blasm, blastx, tblastn and tblastx. It can be accessed at http://www.ncbi.nlm.nih.gov/BLAST/. A description of how to determine sequence identity using this program is available at http://www.ncbi.nlm.nih.gov/BLAST/blast_help.html.

In another aspect of this invention, there is provided a composition for improving a tolerance to a drought stress of a plant, comprising a recombinant plant expression vector which comprises: (a) a nucleotide sequence encoding the amino acid sequence of SEQ ID NO:2 or a nucleotide sequence of SEQ ID NO:1; (b) a promoter which is operatively linked to the nucleotide sequence of (a) and generates RNA molecules in plant cells; and (c) a poly A signal sequence inducing polyadenylation at the 3′-end of the RNA molecules.

The term “operatively linked” refers to functional linkage between a nucleic acid expression control sequence (such as a promoter, signal sequence, or array of transcription factor binding sites) and a second nucleotide sequence, wherein the expression control sequence affects transcription and/or translation of the nucleic acid corresponding to the second sequence.

The vector system of this invention may be constructed in accordance with conventional techniques described in Sambrook et al., Molecular Cloning, A Laboratory Manual, Cold Spring Harbor Laboratory Press (2001).

The suitable promoter in the present invention includes any one commonly used in the art, for example SP6 promoter, T7 promoter, T3 promoter, PM promoter, maize-ubiquitin promoter, Cauliflower mosaic virus (CaMV)-35S promoter, Nopalin synthase (nos) promoter, Figwort mosaic virus 35S promoter, Sugarcane bacilliform virus promoter, commelina yellow mottle virus promoter, photo-inducible promoter of small subunit of Ribulose-1,5-bis-phosphate carboxylase (ssRUBISCO), cytosolic triosphosphate isomerase (TPI) promoter in rice, adenine phosphoribosyltransferase (APRT) or octopine synthase promoter in Arabidopsis.

According to a preferred embodiment, the 3′-non-translated region causing polyadenylation includes that from the nopaline synthase gene of Agrobacterium tumefaciens (NOS 3′ end) (Bevan et al., Nucleic Acids Research, 11(2):369-385(1983)), that from the octopine synthase gene of Agrobacterium tumefaciens, the 3′-end of the protease inhibitor I or II genes from potato or tomato, the CaMV 35S terminator, and OCS (octopine synthase) terminator. Most preferably, the 3′-non-translated region causing polyadenylation in this invention is NOS.

Optionally, the present vector for plants may further carry a reporter molecule (e.g., genes for luciferase and β-glucuronidase). In addition, the vector may contain antibiotic resistant genes as selective markers (e.g., neomycin phosphotransferase gene (nptII) and hygromycin phosphotransferase gene (hpt)).

According to a preferred embodiment, the plant expression vector of this invention is Agrobacterium binary vectors.

The term “binary vector” as used herein, refers to a cloning vector containing two separate vector systems harboring one plasmid responsible for migration consisting of left border (LB) and right border (RB), and another plasmid for target gene-transferring. Any Agrobacterium suitable for expressing the nucleotide of this invention may be used, and for example, the transformation may be carried out using Agrobacterium tumefaciens GV3101.

Introduction of the recombinant vector of this invention into Agrobacterium can be carried out by a large number of methods known to one skilled in the art. For example, particle bombardment, electroporation, transfection, lithium acetate method and heat shock method may be used. Preferably, the electroporation is used.

In still another aspect of this invention, there is provided a plant cell exhibiting improved tolerance to a drought stress, transformed with the composition of this invention.

In still another aspect of this invention, there is provided a plant exhibiting improved tolerance to a drought stress, transformed with the composition of this invention.

To introduce a foreign nucleotide sequence into plant cells or plants may be performed by the methods (Methods of Enzymology, Vol. 153, 1987) known to those skilled in the art. The plant may be transformed using the foreign nucleotide inserted into a carrier (e.g., vectors such as plasmid or virus) or Agrobacterium tumefaciens as a mediator (Chilton et al., Cell, 11: 263-271 (1977)) and by directly inserting the foreign nucleotide into plant cells (Lorz et al., Mol. Genet., 199: 178-182 (1985); the disclosure is herein incorporated by reference). For example, electroporation, microparticle bombardment, polyethylene glycol-mediated uptake may be used in the vector containing no T-DNA region.

Generally, Agrobacterium-mediated transformation is the most preferable (U.S. Pat. Nos. 5,004,863, 5,349,124 and 5,416,011), and the skilled artisan can incubate or culture the transformed cells or seeds to mature plants in appropriate conditions.

The term “plant(s)” as used herein, is understood by a meaning including a plant cell, a plant tissue and a plant seed as well as a mature plant.

The plants applicable of the present method include, but not limited to, most dicotyledonous plants including lettuce, chinese cabbage, potato and radish, and most monocotyledonous plants including rice plant, barley and banana tree. Preferably, the present method can be applied to the plants selected from the group consisting of food crops such as rice plant, wheat, barley, corn, bean, potato, Indian bean, oat and Indian millet; vegetable crops such as Arabidopsis sp., Chinese cabbage, radish, red pepper, strawberry, tomato, watermelon, cucumber, cabbage, melon, pumpkin, welsh onion, onion and carrot; crops for special use such as ginseng, tobacco plant, cotton plant, sesame, sugar cane, sugar beet, Perilla sp., peanut and rape; fruit trees such as apple tree, pear tree, jujube tree, peach tree, kiwi fruit tree, grape tree, citrus fruit tree, persimmon tree, plum tree, apricot tree and banana tree; flowering crops such as rose, gladiolus, gerbera, carnation, chrysanthemum, lily and tulip; and fodder crops such as ryegrass, red clover, orchardgrass, alfalfa, tallfescue and perennial ryograss.

In still another aspect of this invention, there is provided a method for preparing transformed plant exhibiting improved drought stress tolerance, comprising:

(a) introducing the composition of this invention into a plant cell; and

(b) obtaining the transformed plant exhibiting improved drought stress tolerance from the plant cell of (a).

In still another aspect of this invention, there is provided a method for improving a tolerance to a drought stress of a plant, comprising introducing the composition of this invention into a plant cell.

Introducing plant expressing recombinant vectors into a plant cell can be carried out by various methods known to those skilled in the art. Selection of the transformed plant cell can be performed by exposing it to selective agents (e.g., metabolic inhibitors, antibiotics or herbicides). Transformed plant cells stably harboring marker genes which give a tolerance to selective agents are grown and divided in above culture.

The exemplary markers include, but not limited to, hygromycin phosphotransferase (hpt), glyphosate-resistance gene and neomycin phophotransferase (nptII) system.

The methods for developing or regenerating plants from plant protoplasms or various ex-plants are well known to those skilled in the art. The development or regeneration of plants containing the foreign gene of interest introduced by Agrobacterium may be achieved by methods well known in the art (U.S. Pat. Nos. 5,004,863, 5,349,124 and 5,416,011).

According to a concrete embodiment, the plant of this invention is selected from the group consisting of food crops such as rice plant, wheat, barley, corn, bean, potato, Indian bean, oat and Indian millet; vegetable crops such as Arabidopsis sp., Chinese cabbage, radish, red pepper, strawberry, tomato, watermelon, cucumber, cabbage, melon, pumpkin, welsh onion, onion and carrot; crops for special use such as ginseng, tobacco plant, cotton plant, sesame, sugar cane, sugar beet, Perilla sp., peanut and rape; fruit trees such as apple tree, pear tree, jujube tree, peach tree, kiwi fruit tree, grape tree, citrus fruit tree, persimmon tree, plum tree, apricot tree and banana tree; flowering crops such as rose, gladiolus, gerbera, carnation, chrysanthemum, lily and tulip; and fodder crops such as ryegrass, red clover, orchardgrass, alfalfa, tallfescue and perennial ryograss. More concretely, the plant of this invention is food crops such as rice plant, wheat, barley, corn, bean, potato, Indian bean, oat and Indian millet.

In still another aspect of this invention, there is provided a composition for improving a tolerance to an oxidative stress of a plant, comprising a nucleic acid molecule which inhibits an expression of a nucleotide sequence encoding the amino acid sequence of SEQ ID NO:2.

According to a concrete embodiment, the nucleotide sequence comprises the nucleotide sequence of SEQ ID NO:1 or the nucleotide sequence complementary to SEQ ID NO:1.

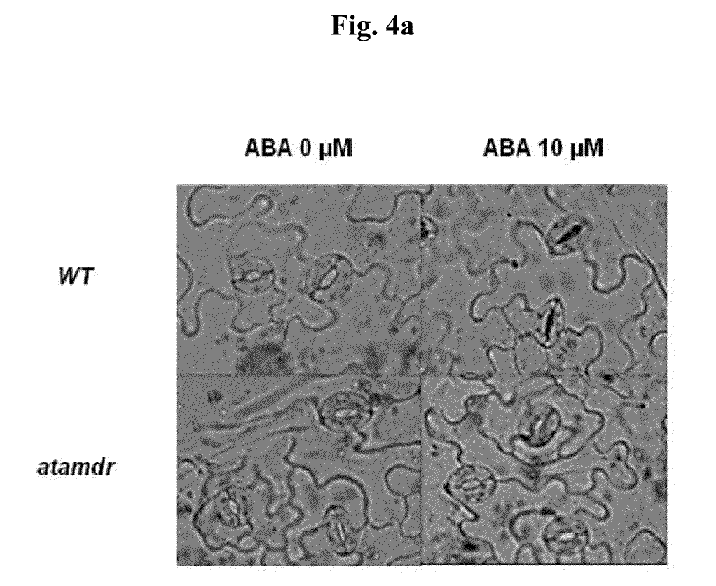

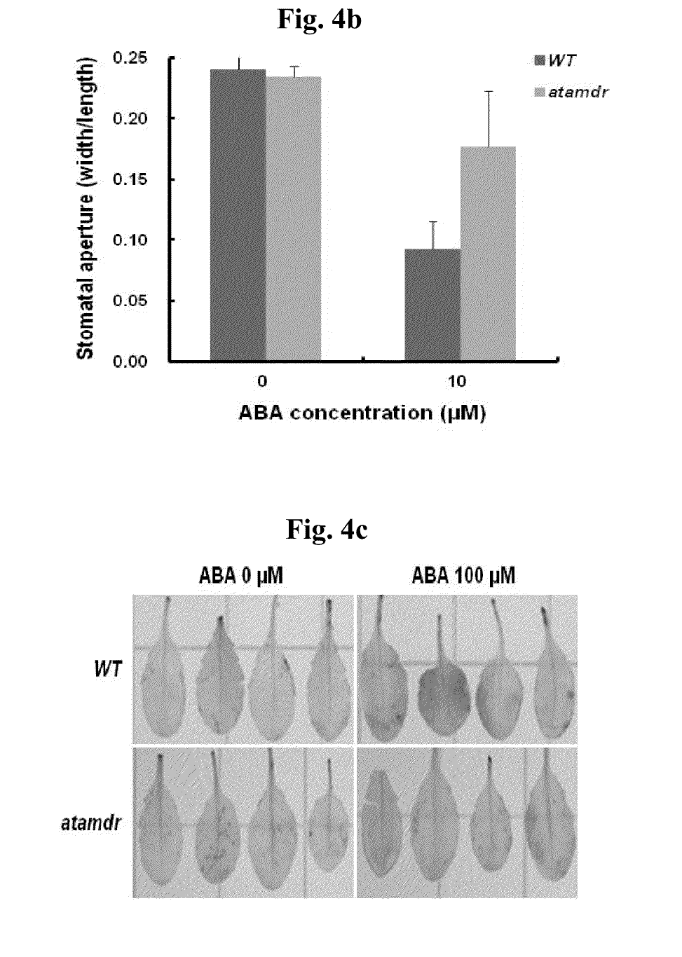

According to the present invention, inhibition of the nucleotide sequence of this invention significantly delayed H₂O₂ accumulation and therefore confers resistance to an oxidative stress (FIG. 4c).

In still another aspect of this invention, there is provided a method for improving a tolerance to an oxidative stress of a plant, comprising introducing the composition of this invention into a plant cell.

As the method and composition for improving a tolerance to an oxidative stress adopt the nucleotide sequence encoding the amino acid sequence of SEQ ID NO:2 or a nucleotide sequence of SEQ ID NO: 1ss of a plant which are mentioned above, they are omitted herein to avoid excessive overlaps.

The terms “inhibiting an expression” used herein refers to any modification of nucleotide sequence that leads to loss or reduce of target gene function. Concretely, it refers to reducing target gene expression to an undetectable or insignificant level.

According to a concrete embodiment, the nucleic acid molecule is T-DNA, siRNA, shRNA, miRNA, ribozyme, PNA (peptide nucleic acids) or antisense oligonucleotide. More concretely, the present nucleic acid molecule is T-DNA.

The term “siRNA” used herein refers to a short double strand RNA that enables to mediate RNA interference via cleavage of mRNA. The siRNA of the present invention may consist of a sense RNA strand having a sequence corresponding to a target gene and an antisense RNA strand having a sequence complementary to the target gene. The siRNA to inhibit expression of a target gene provides effective gene knock-down method or gene therapy method.

The siRNA of this invention is not restricted to a RNA duplex of which two strands are completely paired and may comprise non-paired portion such as mismatched portion with non-complementary bases and bulge with no opposite bases. The overall length of the siRNA is 10-100 nucleotides, preferably 15-80 nucleotides, and more preferably, 20-70 nucleotides. The siRNA may comprise either blunt or cohesive end so long as it enables to inhibit the target gene expression via RNAi effect. The cohesive end may be prepared in 3′-end overhanging structure or 5′-end overhanging structure. The base number protruded is not particularly limited, for example 1-8 bases, preferably 2-6 bases. In addition, siRNA may comprise low molecular weight RNA (for example, tRNA, rRNA, natural RNA molecule such as viral RNA or artificial RNA molecule) in the protruded portion of one end to the extent that it enables to maintain an effect on the inhibition of target gene expression. The terminal structure of siRNA is not demanded as cut structure at both ends, and one end portion of double strand RNA may be stem-and-loop structure linked by a linker RNA. The length of linker is not restricted where it has no influence on the pair formation of the stem portion.

The term “shRNA” used herein refers to a single strand nucleotide consisting of 50-70 bases, and forms stem-loop structure in vivo. Long RNA of 19-29 nucleotides is complementarily base-paired at both directions of loop consisting of 5-10 nucleotides, forming a double-stranded stem.

The term “miRNA (microRNA)” functions to regulate gene expression and means a single strand RNA molecule composed of 20-50 nucleotides in full-length, preferably 20-45 nucleotides, more preferably 20-40 nucleotides, much more preferably 20-30 nucleotides and most preferably, 21-23 nucleotides. The miRNA is an oligonucleotide which is not expressed intracellularly, and forms a short stem-loop structure. The miRNA has a whole or partial complementarity to one or two or more mRNAs (messenger RNAs), and the target gene expression is suppressed by the complementary binding of miRNA to the mRNA thereof.

The term used herein “ribozyme” refers to a RNA molecule having an activity of an enzyme in itself which recognizes and restricts a base sequence of a specific RNA. The ribozyme consists of a binding portion capable of specifically binding a base sequence complementary to a transfer RNA strand and an enzymatic portion to cut target RNA.

The term “PNA (peptide nucleic acid)” used herein refers to a molecule having the characteristics of both nucleic acid and protein, which is capable of complementarily binding to DNA or RNA. PNA was first reported in 1999 as similar DNA in which nucleobases are linked via a peptide bond (Nielsen P E, Egholm M, Berg R H, Buchardt 0, “Sequence-selective recognition of DNA by strand displacement with a thymine-substituted polyamide”, Science 1991, Vol. 254: pp 1497-1500). PNA is absent in the natural world and artificially synthesized through a chemical method. PNA is reacted with a natural nucleic acid having a complementary base sequence through hybridization response, forming double strand. In the double strand with the same length, PNA/DNA and PNA/RNA double strand are more stable than DNA/DNA and DNA/RNA double strand, respectively. The form of repeating N-(2-aminoethyl)-glycine units linked by amide bonds is commonly used as a basic peptide backbone. In this context, the backbone of peptide nucleic acid is electrically neutral in comparison to that of natural nucleic acids having negative charge. Four bases of nucleic acid present in PNA are almost the same to those of natural nucleic acid in the respect of spatial size and distance between nucleobases. PNA has not only a chemical stability compared with natural nucleic acid, but also a biological stability due to no degradation by a nuclease or protease.

The term “antisense oligonucleotide” used herein is intended to refer to nucleic acids, preferably, DNA, RNA or its derivatives, that are complementary to the base sequences of a target mRNA, characterized in that they bind to the target mRNA and interfere its translation to protein. The antisense oligonucleotide of the present invention refers to DNA or RNA sequences which are complementary to a target mRNA, characterized in that they bind to the target mRNA and interfere its translation to protein, translocation into cytoplasm, maturation or essential activities to other biological functions. The length of antisense nucleic acids is in a range of 6-100 nucleotides and preferably 10-40 nucleotides.

The antisense oligonucleotides may be modified at above one or more positions of base, sugar or backbone to enhance their functions [De Mesmaeker, et al., Curr Opin Struct Biol., 5(3): 343-55 (1995)]. The oligonucleotide backbone may be modified with phosphothioate, phosphotriester, methyl phosphonate, single chain alkyl, cycloalkyl, single chain heteroatomic, heterocyclic bond between sugars, and so on. In addition, the antisense nucleic acids may include one or more substituted sugar moieties. The antisense oligonucleotides may include a modified base. The modified base includes hypoxanthine, 6-methyladenine, 5-me pyrimidine (particularly, 5-methylcytosine), 5-hydroxymethylcytosine (HMC), glycosyl HMC, gentobiosyl HMC, 2-aminoadenine, 2-thiouracil, 2-thiothymine, 5-bromouracil, 5-hydroxymethyluracil, 8-azaguanine, 7-deazaguanine, N6(6-aminohexyl)adenine, 2,6-diaminopurine, and so forth.

The term “T-DNA” used herein refers to a DNA fragment as a transfer DNA in Ti (tumor-inducing) plasmid of Agrobacterium sp., which is transferred into a nucleus of a host plant cell. A 25 bp repeat sequence is present in both termini of T-DNA, and DNA transfer proceeds at the direction from a left border to a right border. A bacterial T-DNA with about 20,000 in length destroys a target gene by insertion, resulting in insertional muatagenesis. In addition to mutation, inserted T-DNA sequence may label a target gene. AtAMDR expression of the mutant plant used in the present invention is suppressed by T-DNA insertions. T-DNA were mapped to the first exon of AtAMDR mutant, which was verified by PCR amplification using the T-DNA border primer and primers annealing to sites upstream and downstream of the T-DNA insertion site.

In still another aspect of this invention, there is provided a composition for improving a germination rate of a plant, comprising a nucleic acid molecule which inhibits an expression of a nucleotide sequence encoding the amino acid sequence of SEQ ID NO:2.

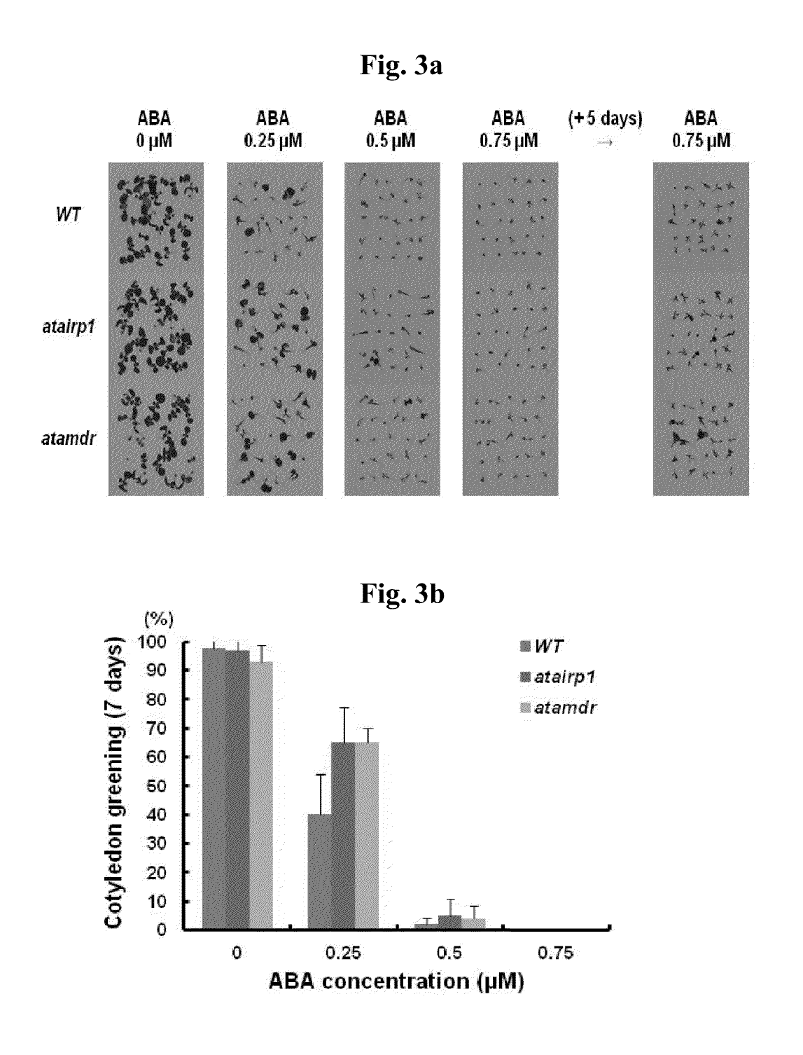

According to the present invention, inhibiting the expression of the nucleotide sequence of the present invention reduces sensitivity to ABA resulting in germination rate improvement (FIGS. 3a-3b).

According to a concrete embodiment, the nucleotide sequence comprises the nucleotide sequence of SEQ ID NO:1 or the nucleotide sequence complementary to SEQ ID NO:1.

According to a concrete embodiment, the nucleic acid molecule is T-DNA, siRNA, shRNA, miRNA, ribozyme, PNA (peptide nucleic acids) or antisense oligonucleotide. More concretely, the present nucleic acid molecule is T-DNA.

In still another aspect of this invention, there is provided a method for improving a germination rate of a plant, comprising introducing the composition of this invention into a plant cell.

The features and advantages of the present invention will be summarized as follows:

(a) The present invention provides a composition for improving a tolerance to drought and oxidative stress of a plant, a plant transformed with the composition, a method for preparing a plant transformed with the composition and a method for improving a tolerance to a drought and oxidative stress.

(b) The present invention provides a composition and method for a germination rate of a plant.

(c) The nucleotide sequences of the present invention are involved in the drought and oxidative stress-tolerance and germination capacity, therefore may be effectively used for cultivating the plants with enhanced tolerance against abiotic stress or novel function of rapid growing or bolting.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Materials and Methods

Isolation of the Genes

The present inventors have isolated the AtAMDR gene inducible by ABA hormone and abiotic stresses from cDNAs of Arabidopsis thaliana. 10-day-old Arabidopsis thaliana seedlings were grinded with liquid nitrogen in mortar. The powders were added with 2 ml of an extraction buffer (4 M guanidine-HCl 20 mM, 10 mM EDTA, 10 mM EGTA (USB), 0.5% Sarkosyl (SIGMA), pH 9) and β-mercaptoethanol (SIGMA-ALDRICH) per 1 g of the powder for extraction. The extraction resultant was transferred to new conical tube, suspended with an equal volume of PCI (phenol:chloroform:isoamyl alcohol=25:24:1), vortexed for 5 min and centrifuged at 3,500 rpm for 25 min (Hanil centrifuge, HA-1000-3). After centrifugation, the upper organic solvent phase was removed. The extract was resuspended with an equal volume of PCI, vortexed and centrifuged twice. Then, the lower aqueous phase was undergone twice ethanol precipitation and once LiCl precipitation to isolate RNA. RNA was quantified. Single-strand cDNA was synthesized by using 2 μg of RNA with oligo dT primer and MMLV reverse transcriptase (Fermentas). PCR was conducted in the final volume of 50 μL containing 20 ng of cDNA as a template, 10 pmole of each of two types of primers, 5 μL of 10×Taq polymerase buffer (Takara), 8 μL of dNTPs (each of 1.25 mM) and 1 unit of Taq DNA polymerase (Takara). The tube containing the reaction mixture was placed in Perkin Elmer DNA thermal cycler. The sequences of primers used in this Example are as follows: 5′-ATGTCAAGCAGTCGAAATACCC-3′ (AtAMDR ORF FW: SEQ ID NO: 3) and 5′-TTAGTAATCAAAAGGCCAGCCAC-3′ (AtAMDR ORF RV: SEQ ID NO: 4). The reaction mixture was denatured for 2 min at 98° C. and subjected to 30 cycles of 10 sec at 98° C., 5 sec at 55° C. and 1 min at 72° C. After 30 cycles, polymerization was further performed at 72° C. for 2 min. Then, the AtAMDR gene amplification was verified by using electrophoresis method. In addition, the DNA was confirmed by sequencing 3′ (SEQ ID NO: 1, AtAMDR CDS nucleotide sequence; SEQ ID NO: 2, AtAMDR amino acid sequence).

Plant Growth Conditions and Sampling

Seeds of the AtAMDR, AtAIRP1 and AtAIRP2 knock-out mutants [AtAMDR seed number: Salk_{—}037121; AtAIRP1_seed number: Salk_{—}110094; AtAIRP2_seed number: Salk_{—}005082(atairp2-2)] which are T-DNA insertion lines were purchased from SIGNAL Salk Institute Genomic Analysis Laboratory (http://signal.salk.edu/).

The seeds of the knock-out mutants and the wild type Arabidopsis thaliana were soaked in 30% bleach solution (Yuhan-clorox) and 0.025% Triton X-100 for 10 min, and washed 10 times with sterilized water. The treated seeds were grown on MS (Murashige and Skoog) medium (Duchefa Biochemie) that contained 3% sucrose, B5 vitamin (12 mg/L) and 0.8% agar (pH 5.7) in a growth chamber for 2 weeks (under a condition of continuous light). Where green whole plants of light condition were used as materials, seeds were grown on soil of Sunshine MIX #5 (Sun GroHorticulture) in a growth chamber for 3 weeks (under a condition of continuous light).

Treatments of Stresses (Salt, Low-Temperature and Drought)

In order to determine expressions of the AtAMDR gene to drought stress, the wild type Arabidopsis thaliana seedlings which were grown on medium for 2 weeks were exposed in the air, and sampled after 1 hour and 2 hours. In order to determine expressions of the AtAMDR gene to salt stress, the wild type Arabidopsis thaliana seedlings which were grown on medium for 2 weeks were treated with 300 mM sodium chloride, and sampled after 1 hour and 3 hours. In order to determine expressions of the AtAMDR gene to low-temperature stress, the wild type Arabidopsis thaliana seedlings which were grown on medium for 2 weeks were incubated at 4° C. for 6 hours and 12 hours, and sampled. In addition, the Arabidopsis thaliana seedlings which were grown for 2 weeks were treated with 100 μM of ABA (SIGMA), and sampled after 1 hour and 3 hours.

The sampled tissues were grinded with liquid nitrogen in mortar. The powders were added with 2 ml of an extraction buffer (4 M guanidine-HCl 20 mM, 10 mM EDTA, 10 mM EGTA (USB), 0.5% Sarkosyl (SIGMA), pH 9) per 1 g of the powder and β-mercaptoethanol (SIGMA-ALDRICH) to extract. The extract was transferred to conical tube, suspended with an equal volume of PCI (phenol:chloroform:isoamyl alcohol=25:24:1), vortexed for 5 min and centrifuged at 3,500 rpm for 25 min (Hanil centrifuge, HA-1000-3). After centrifugation, the supernatant of upper organic solvent phase was removed. The extract was resuspended with an equal volume of PCI, vortexed and centrifuged. The extract was performed twice with the process described above. Then, the lower aqueous phase was performed twice with ethanol precipitation and once with LiCl precipitation to isolate RNA.

Quantitative Reverse Transcriptase Polymerase Chain Reaction (RT-PCR)

Total RNA was isolated from leaves of the mutants and the wild-type plants. Single-strand cDNA was synthesized by using 2 μg of RNA with oligo dT primer and MMLV reverse transcriptase (Fermentas). PCR was conducted in the final volume of 50 μL containing 20 ng of cDNA as a template, 10 pmole of each of two types of primers, 5 μL of 10×Taq polymerase buffer (Intron), 8 μL of dNTPs (each of 1.25 mM) and 1 unit of Taq DNA polymerase (Intron). The tube containing the reaction mixture was placed in Perkin Elmer DNA thermal cycler. The reaction mixture was denatured for 5 min at 94° C. and subjected to 30 cycles of 30 sec at 94° C., 30 sec at 50° C. and 30 sec at 72° C. After 30 cycles, polymerization was further performed at 72° C. for 1 min. Then, the PCR products were stored at −20° C. in a freezer. The sequences of primers used in this Example are shown in Table 1.

TABLE 1
Primers used in RT-PCR

| primer | sequence |
|---|---|
| AtAMDR ORF FW | 5′-ATGTCAAGCAGTCGAAATACCC-3′ |
| (SEQ ID No: 3) | |
| AtAMDR ORF RV | 5′-TTAGTAATCAAAAGGCCAGCCAC-3′ |
| (SEQ ID No: 4) | |
| AtAMDR FW1 | 5′-GTTTTTGTTCAATCGCTCTGC-3′ |
| (SEQ ID No: 5) | |
| AtAMDR FW2 | 5′-ATAGAGAGTGAAGCACAAGGC-3′ |
| (SEQ ID No: 6) | |
| AtAMDR RV1 | 5′-TGATCTTTGGTGGTCAAGTC-3′ |
| (SEQ ID No: 7) | |
| AtAMDR RV2 | 5′-CAGCTTTCAGCTGGCACTAC-3′ |
| (SEQ ID No: 8) | |
| Rab18 FW | 5′-GCGTCTTACCAGAACCGTCC-3′ |
| (SEQ ID No: 9) | |
| Rab18 RV | 5′-CCCTTCTTCTCGTGGTGC-3′ |
| (SEQ ID No: 10) | |
| RD29A FW | 5′-CAGGTGAATCAGGAGTTGTT-3′ |
| (SEQ ID No: 11) | |
| RD29A RV | 5′-CCGGAAATTTATCCTCTTCT-3′ |
| (SEQ ID No: 12) | |
| UBC10 FW | 5′-TGGATATGGCGTCGAAGC-3′ |
| (SEQ ID No: 13) | |
| UBC10 RV | 5′-GTGGGATTTTCCATTTAGCC-3′ |
| (SEQ ID No: 14) | |

Extraction of Genomic DNA of Mutants Inserted with T-DNA and Acquisition of Homozygous Mutant

The seeds of the wild type Arabidopsis thaliana and knock-out mutants were grown on soil for 2 weeks and their leave were sampled. The leaves were grinded with liquid nitrogen in mortar. The powders were added with 700 ml of CTAB buffer (2% CTAB, 100 mM Tris pH 8, 20 mM EDTA, 1.4 M NaCl, 2% PVP), mixed and heated at 65° C. for 10 min. The resultants were added to 200 ml of chloroform, mixed and centrifuged. After centrifugation, the supernatant was removed. The resultant was mixed with isopropanol to precipitate DNA. The precipitate was washed with 70% ethanol, dried. The obtained genomic DNA was dissolved in water to use. Genotyping PCR was performed using T-DNA border primer (LB_{—}6313R) and primers annealing to sites upstream and downstream of the T-DNA insertion site.

TABLE 2
Primers used in Genotyping PCR and RT-PCR

| primer | sequence |
|---|---|
| LB_6313R | 5′-GAGCTGCTATACACTGATCTGAG-3′ |
| (SEQ ID No: 15) | |
| AtAMDR FW1 | 5′-GTTTTTGTTCAATCGCTCTGC-3′ |
| (SEQ ID No: 5) | |
| AtAMDR FW2 | 5′-ATAGAGAGTGAAGCACAAGGC-3′ |
| (SEQ ID No: 6) | |
| AtAMDR RV1 | 5′-TGATCTTTGGTGGTCAAGTC-3′ |
| (SEQ ID No: 7) | |
| AtAMDR RV2 | 5′-CAGCTTTCAGCTGGCACTAC-3′ |
| (SEQ ID No: 8) | |

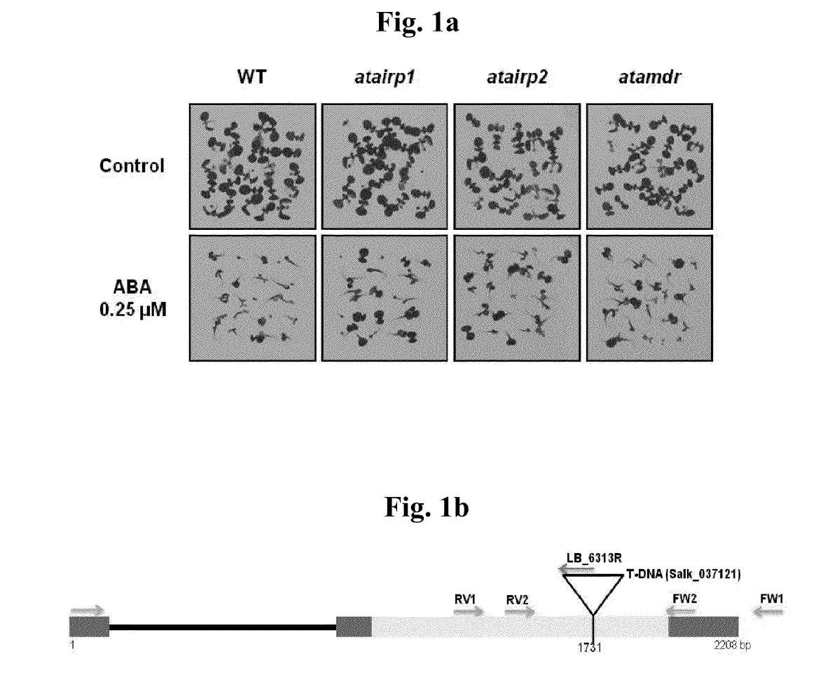

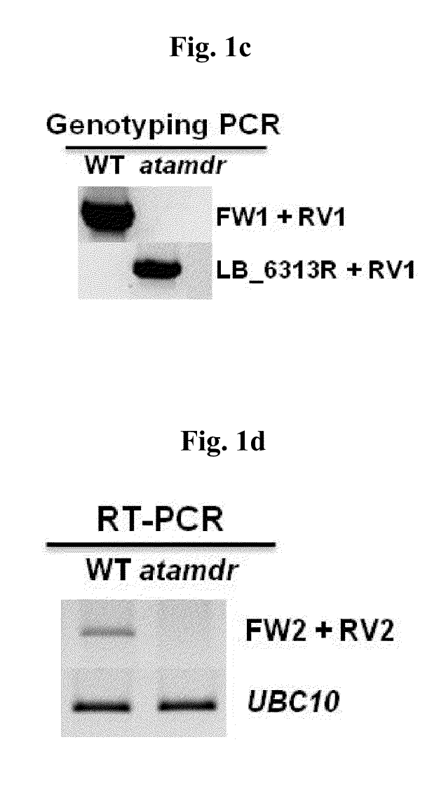

It was revealed that the T-DNA insertions were mapped to the first exon of AtAMDR mutant, which was verified by PCR amplification using the T-DNA border primer and primers annealing to sites upstream and downstream of the T-DNA insertion site. In addition, the suppression of AtAMDR expression was analyzed by RT-PCR with the extracted RNA from the knock-out mutant (FIGS. 1c-1d).

Comparison of Germination Rates

For comparing phenotypes to ABA hormone, seeds obtained from the wild-type, the AtAIRP1 knock-out mutant and the AtAMDR knock-out mutant were grown on medium supplemented with different concentrations (0, 0.25, 0.5 and 0.75 μM) of ABA hormone for 7 days, and their germination degrees were then measured.

Stomatal Aperture Measurement

Mature leaves of light-grown, 4-week-old wild-type and atamdr mutant plants were used for the observation of mature stomata. Detached leaves were incubated in stomatal opening solution (10 mM MES-KOH, 50 mM CaCl2, 10 mM KCl) for 2 h and then transferred to stomatal opening solution supplemented with 0 or 10 μM ABA for 2 h. Subsequently, epidermal strips were mounted on glass slides and observed with a light microscope.

H₂O₂ Production of Atamdr Mutant Plants

To investigate the accumulation of H₂O₂, light-grown, 4-week-old mature leaves of wild-type (WT), atamdr plants were incubated in stomatal opening solution for 2 h and treated with 0 or 100 μM ABA. Leaves were transferred to 100 μg/mL DAB (3,3′-diaminobenzidine-4HCl) solution overnight, and washed and fixed by 96% ethanol. Leaves were observed after complete chlorosis (Luis David Maldonado-Bonilla et al.; Eur J Plant Pathol. 121:439449, 2008).

Measurement of Sensitivity to Drought Stress in Mature Plants

Light-grown, 2.5-week-old wild-type and atamdr mutant plants were further grown for 13 d without watering. Plants were rewatered, and surviving plants were counted 3 d after rewatering.

Results

Identification and Characterization of the Mutants Inserted with AtAMDR T-DNA

ABA-insensitive mutant plants were screened at the germination stage. Wild-type and various knock-out mutants were cultured in MS media containing 0 μM and 0.25 μM ABA for 7 days. AtAIRP1, AtAIRP2 and AtAMDR knock-out mutants showed reduced sensitivity to ABA, indicating that these mutants have improved germination rates (FIG. 1a).

The AtAMDR Gene Expression after Stress Treatments (Salt, Low-Temperature and Drought)

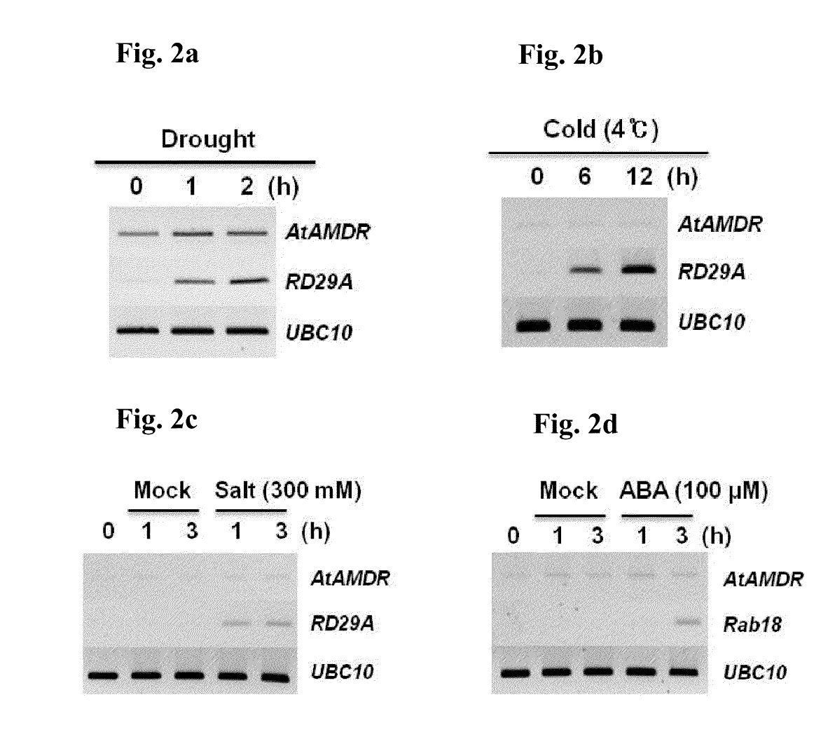

The AtAIRP2 gene expressions in various abiotic stresses were analyzed by RT-PCR. After treatments of drought (1 hour and 2 hours) (FIG. 2a), low-temperature (6 hours and 12 hours) (FIG. 2b), 300 mM salt solution (1 hour and 3 hours) (FIG. 2c) and 300 μM ABA hormone (1 hour and 3 hours) (FIG. 2d), RNA from each of samples was extracted to analyze the AtAIRP2 gene expression patterns. As a result, it was determined that the gene expression levels were increased by drought stress treatments than those of non-stress treatments, whereas low-temperature and salt stresses did not affect on the gene expression. In addition, AtAIRP2 gene expression was not affected by ABA treatment in atamdr mutant.

Comparison of Germination Rates

Wild-type and AtAIRP1, AtAMDR knock-out mutants were cultured in media containing 0 μM, 0.25 μM, 0.5 μM and 0.75 μM of ABA for 7 days. AtAIRP1 and AtAMDR knock-out mutants showed reduced sensitivity to ABA (FIG. 3a), and AtAIRP1 and AtAMDR knock-out mutants exhibited improved germination rates (FIG. 3b).

Stomatal Aperture Measurement

Mature leaves of light-grown, 4-week-old wild-type and atamdr mutant plants were detached and incubated in stomatal opening solution and then transferred to stomatal opening solution supplemented with various concentrations of ABA (0 and 10 μM) for 2 h. Subsequently, epidermal strips were observed with a light microscope. As results, atamdr mutant plants exhibited ABA-insensitivity and the stomata of atamdr mutant still remained open in the presence of higher concentrations of ABA (FIGS. 4a-4b). Stomatal aperture of wild-type plants treated with 10 μm ABA was approximately 0.09 which was 2-fold closed than that of mutant, while stomatal aperture of atamdr mutant treated with 10 μm ABA was approximately 0.175, which was 1.94-fold compared to that of wild-type plants.

H₂O₂ Production of Atamdr Mutant Plants

To investigate the accumulation of H₂O₂, light-grown, 4-week-old mature leaves of wild-type and atamdr plants were treated with 0 or 100 μM ABA, and stained with 100 ng/mL DAB (3,3′-diaminobenzidine-4HCl) solution in dark. As results, the accumulation of H₂O₂ was drastically arisen in wild-type, while those in atamdr plants was significantly delayed (FIG. 4c). These results suggest that H2O2 is involved in senility and H₂O₂ accumulation in plants, and inhibition of AtAMDR expression leads to delay of H₂O₂ accumulation and therefore confers resistance to an oxidative stress.

Measurement of Sensitivity to Drought Stress in Mature Plants

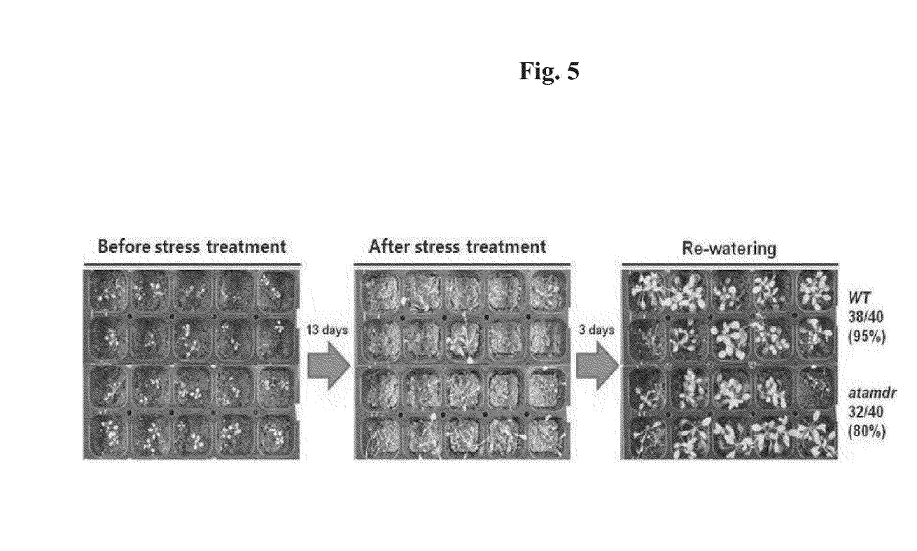

To investigate the tolerance to drought stress, light-grown, 2.5-week-old wild-type and atamdr mutant plants were further grown for 13 d without watering. Plants were rewatered, and surviving plants were counted 3 d after rewatering. As results, 95% of the wild-type plants were able to resume their growth and survived, whereas only 80% of the mutant plants survived after rewatering (FIG. 5), indicating that loss-of-function mutant plants were more susceptible to water deficit than wild-type plants.

Having described a specific embodiment of the present invention, it is to be understood that variants and modifications thereof falling within the spirit of the invention may become apparent to those skilled in this art, and the scope of this invention is to be determined by appended claims and their equivalents.

What is claimed is:

1. A method for improving a tolerance to a drought stress of a plant, the method comprising:
introducing a nucleotide sequence encoding the amino acid sequence of SEQ ID NO:2 into a cell of the plant; and
obtaining a transgenic plant exhibiting improved tolerance to a drought stress from the cell of the plant.

2. The method according to claim 1, wherein the nucleotide sequence comprises the nucleotide sequence of SEQ ID NO:1.

3. The method according to claim 1, wherein the nucleotide sequence is contained in a recombinant plant expression vector which comprises:
the nucleotide sequence;
a promoter which is operatively linked to the nucleotide sequence and generates RNA molecules in plant cells; and
a poly A signal sequence inducing polyadenylation at the 3′-end of the RNA molecules.

4. A plant cell exhibiting improved tolerance to a drought stress, transformed with the method according to claim 1.

5. A plant exhibiting improved tolerance to a drought stress, transformed with the method according to claim 1.

6. A method for improving a tolerance to an oxidative stress of a plant, the method comprising:
introducing a nucleic acid molecule which inhibits an expression of a nucleotide sequence encoding the amino acid sequence of SEQ ID NO:2 into a cell of the plant; and
obtaining a transgenic plant exhibiting improved tolerance to a oxidative stress from the cell of the plant.

7. The method according to claim 6, wherein the nucleotide sequence comprises the nucleotide sequence of SEQ ID NO:1 or the nucleotide sequence complementary to SEQ ID NO:1.

8. The method according to claim 6, wherein the nucleic acid molecule is T-DNA, siRNA, shRNA, miRNA, ribozyme, PNA (peptide nucleic acids) or antisense oligonucleotide.

9. The method according to claim 8, wherein the nucleic acid molecule is T-DNA.

10. A method for improving a germination rate of a plant, the method comprising:
introducing a nucleic acid molecule which inhibits an expression of a nucleotide sequence encoding the amino acid sequence of SEQ ID NO:2 into a cell of the plant; and
obtaining a transgenic plant exhibiting improved germination rate from the cell of the plant.

11. The method according to claim 10, wherein the nucleotide sequence comprises the nucleotide sequence of SEQ ID NO:1 or the nucleotide sequence complementary to SEQ ID NO:1.

12. The method according to claim 10, wherein the nucleic acid molecule is T-DNA, siRNA, shRNA, miRNA, ribozyme, PNA (peptide nucleic acids) or antisense oligonucleotide.

13. The method according to claim 12, wherein the nucleic acid molecule is T-DNA.


SEQUENCE LISTING (16 sequences)

SEQ ID NO: 1 - DNA, 987 bp, Artificial Sequence
  AtAMDR_CDS
atgtcaagcagtcgaaatacccactggtgtcacagatgtcagcgtgctgtccgccttcacggccaagagcctgtatgcttttattgcggaggtggatttgttgaagaacttgatatggctcaagccagcccctttgatatgtttagatcccacaggggtgttgtagaacgtgatcagacttttgatctcatggatgctttctctgtgtttatgaggaaccgcttagctgaaaggagccacgacagagaaatcagaggaagaaccatcagttcaggtcctgaaaactttcctggtctggcccctttgttgatctttggtggtcaagtcccttatagactaactggcgacaatgcagtcgaagccctcttcaatggcggctcccctggcattggcatcacacgtggtaacaccggcgactacttcttcggtcccggccttgaagaattgttcgagcagctttcagctggcactactcgccgaggcccaccacccgcaccgagatcagcaatagacgcattgccaactatcaagatcgcgcagaggcatcttaggtcatcggactcgaattgtcccgtgtgcaaagacgaattcgaactgggatcagaagcgaaacagatgccgtgtaaccacatctatcattctgactgcattgtcccgtggctggttcagcacaactcttgcccggtctgtcgccaagagctaccatcagctagcggaccttcaagcagtcaaaacagaaccacccctaccagaaactacagaagcagtagtagtagtagtagtagtaacagtcgtgagaacgggaatgaaagaaggaatcctttctcttccttctggccattccgttcgtcaggttcaagctcaagctccactcaaaaccgtggaggcacaagaaactcggatacaagcgatgagaaccataactaccatcaacagcaacatcaacaatcatatatgggttacagtggctggccttttgattactaa

SEQ ID NO: 2 - protein, 328 aa, Artificial Sequence
  AtAMDR_amino acid
MSSSRNTHWCHRCQRAVRLHGQEPVCFYCGGGFVEELDMAQASPFDMFRSHRGVVERDQTFDLMDAFSVFMRNRLAERSHDREIRGRTISSGPENFPGLAPLLIFGGQVPYRLTGDNAVEALFNGGSPGIGITRGNTGDYFFGPGLEELFEQLSAGTTRRGPPPAPRSAIDALPTIKIAQRHLRSSDSNCPVCKDEFELGSEAKQMPCNHIYHSDCIVPWLVQHNSCPVCRQELPSASGPSSSQNRTTPTRNYRSSSSSSSSNSRENGNERRNPFSSFWPFRSSGSSSSSTQNRGGTRNSDTSDENHNYHQQQHQQSYMGYSGWPFDY

SEQ ID NO: 3 - DNA, 22 bp, Artificial Sequence
  AtAMDR ORF FW
atgtcaagcagtcgaaataccc

SEQ ID NO: 4 - DNA, 23 bp, Artificial Sequence
  AtAMDR ORF RV
ttagtaatcaaaaggccagccac

SEQ ID NO: 5 - DNA, 21 bp, Artificial Sequence
  AtAMDR FW1
gtttttgttcaatcgctctgc

SEQ ID NO: 6 - DNA, 21 bp, Artificial Sequence
  AtAMDR FW2
atagagagtgaagcacaaggc

SEQ ID NO: 7 - DNA, 20 bp, Artificial Sequence
  AtAMDR RV1
tgatctttggtggtcaagtc

SEQ ID NO: 8 - DNA, 20 bp, Artificial Sequence
  AtAMDR RV2
cagctttcagctggcactac

SEQ ID NO: 9 - DNA, 20 bp, Artificial Sequence
  Rab18 FW
gcgtcttaccagaaccgtcc

SEQ ID NO: 10 - DNA, 18 bp, Artificial Sequence
  Rab18 RV
cccttcttctcgtggtgc

SEQ ID NO: 11 - DNA, 20 bp, Artificial Sequence
  RD29A FW
caggtgaatcaggagttgtt

SEQ ID NO: 12 - DNA, 20 bp, Artificial Sequence
  RD29A RV
ccggaaatttatcctcttct

SEQ ID NO: 13 - DNA, 18 bp, Artificial Sequence
  UBC10 FW
tggatatggcgtcgaagc

SEQ ID NO: 14 - DNA, 20 bp, Artificial Sequence
  UBC10 RV
gtgggattttccatttagcc

SEQ ID NO: 15 - DNA, 23 bp, Artificial Sequence
  LB_6313R
gagctgctatacactgatctgag

SEQ ID NO: 16 - DNA, 1468 bp, Artificial Sequence
  AtAMDR_cDNA
gtgcgttcgtagagactgtaataaagacggagagattcttctagagtcagttcttcttcttcatcctcttcttccccccaaatcctctctcgctagggtttctcattccgatccataattctagatgtcgaggtttttaactgagacctgtcaaataagctaaagcttcttctaagagcttcaaggaaagcattgcattgttgtggtcttgtttgtgtccacagaaggaaagaagagtgagaagctcaggatgtcaagcagtcgaaatacccactggtgtcacagatgtcagcgtgctgtccgccttcacggccaagagcctgtatgcttttattgcggaggtggatttgttgaagaacttgatatggctcaagccagcccctttgatatgtttagatcccacaggggtgttgtagaacgtgatcagacttttgatctcatggatgctttctctgtgtttatgaggaaccgcttagctgaaaggagccacgacagagaaatcagaggaagaaccatcagttcaggtcctgaaaactttcctggtctggcccctttgttgatctttggtggtcaagtcccttatagactaactggcgacaatgcagtcgaagccctcttcaatggcggctcccctggcattggcatcacacgtggtaacaccggcgactacttcttcggtcccggccttgaagaattgttcgagcagctttcagctggcactactcgccgaggcccaccacccgcaccgagatcagcaatagacgcattgccaactatcaagatcgcgcagaggcatcttaggtcatcggactcgaattgtcccgtgtgcaaagacgaattcgaactgggatcagaagcgaaacagatgccgtgtaaccacatctatcattctgactgcattgtcccgtggctggttcagcacaactcttgcccggtctgtcgccaagagctaccatcagctagcggaccttcaagcagtcaaaacagaaccacccctaccagaaactacagaagcagtagtagtagtagtagtagtaacagtcgtgagaacgggaatgaaagaaggaatcctttctcttccttctggccattccgttcgtcaggttcaagctcaagctccactcaaaaccgtggaggcacaagaaactcggatacaagcgatgagaaccataactaccatcaacagcaacatcaacaatcatatatgggttacagtggctggccttttgattactaaaagtctaaaagtgagctaaaacaaatctttggtctcactttggtctctttacttaaatatgcttgtgattctgcatatgccttgtgcttcactctctatctctctcactctcttttatcaacataaataatggagaactgtatttgttactgtagattttctttctctgttaaaaacgagttcatcattttctatatgctttgttaaatatgtcattttagttgggttttt